(19) United States
(12) Patent Application Publication
(10) Pub. No.: US 2024/0161907 A1
(43) Pub. Date: May 16, 2024

(54) SYSTEM FOR GENERATING AND EDITING ULTRASOUND IMAGES

(71) Applicant: HUMANSCAPE INC., Seoul (KR)

(72) Inventor: Jong Young JUNG, Seoul (KR)

(21) Appl. No.: 18/462,584

(22) Filed: Sep. 7, 2023

Related U.S. Application Data
(63) Continuation of application No. PCT/KR2023/008434, filed on Jun. 19, 2023.

(30) Foreign Application Priority Data
Nov. 11, 2022 (KR) 10-2022-0150289

(51) Int. Cl. G16H 30/40; G06T 7/00; G06T 11/00

(52) U.S. Cl. CPC G16H 30/40; G06T 7/0012; G06T 11/00; G06T 2207/10132

(57) ABSTRACT

A system for generating and editing ultrasound images according to the present disclosure may include a memory and a processor. The processor may be configured to: based on a probe contacting a skin of a pregnant woman, transmit a signal to an ultrasound device to request that imaging start; and based on that an image signal acquired from the ultrasound device an amount of pixel change on an imaging screen, determine whether the imaging screen is in a paused state. The processor may be configured to: take a snapshot on the imaging screen at a time when the imaging screen is determined to be in the paused state; and based on movement of the probe, determine whether to stop or resume the imaging. Based on a value of a grey scale on the imaging screen exceeding a specified level, the processor may transmit a signal to the ultrasound device to request that the imaging be terminated, and store, in the memory, images captured from a moment of start of the imaging to a moment of termination of the imaging.

### TECHNICAL FIELD

The following embodiments relate to a system for generating and editing ultrasound images.

### BACKGROUND ART

An ultrasound diagnostic device may refer to a device that radiates ultrasound signals from a subject's body surface toward a specific part inside the body and obtains images of soft tissue layers or blood flow non-invasively based on information from the reflected ultrasound signals (ultrasound echo signals).

Compared to other imaging diagnostic devices such as X-ray diagnostic devices, computerized tomography (CT) scanners, magnetic resonance imaging (MRI), and nuclear medicine diagnostic devices, ultrasound diagnostic devices have the advantages of small size, low cost, real-time display, and high safety because they do not require exposure to X-rays. Because of these advantages, ultrasound diagnostic devices are widely used for cardiac, breast, abdominal, urinary, and gynecologic diagnosis.

The examiner can perform ultrasound diagnosis by holding the probe in one hand and moving the probe in contact with the subject's body surface while operating the control panel with the other hand. Ultrasound images obtained by such ultrasound diagnostics are shown in real time on a display, allowing the examiner to diagnose the condition of the subject.

After capturing an image of the fetus in the mother's womb, the hospital delivers the ultrasound image of the fetus to the mother, who can view the image and inform her family about the development of the fetus.

### PRIOR ART LITERATURE

Patent Documents
(Prior art document 0001) Korean Patent No. 10-1588915
(Prior art document 0002) Korean Patent No. 10-2002408

### DISCLOSURE

### Technical Problem

When taking images of a fetus displayed on a display during an examination, there is no particular editing point, or it is difficult to distinguish important moments. Therefore, all images taken during the examination period can be saved and sent to the user.

In this case, the recorded video is relatively long, thus taking up a relatively large amount of storage capacity. In addition, it takes a long time and a higher cost to send the video to the user through communication due to the large size of the file. The user who receives the video can view the video to understand the condition of the fetus or notify others. However, if the length of the video exceeds a certain time, it may be difficult for the user to immediately understand the condition of the fetus, and the video may be excessively large and not suitable for transmission to others.

Unlike a method of photographing and storing all segments of an examination from the moment the examination starts until user input indicates that the examination is over, a system for generating and editing ultrasound images according to one embodiment may start imaging from the moment the examiner's ultrasound probe contacts the subject to be examined after the examination starts, detect that the examination is over based on grey scale and motion levels in the image, and stop photographing.

The system for generating and editing ultrasound images according to one embodiment may capture images of fetal movement or other moments deemed important by the examiner based on the grey scale and motion level of the images and present them to the user separately.

### Technical Solution

A system for generating and editing ultrasound images according to the present disclosure may include a memory and a processor. The processor may be configured to: based on a probe contacting a skin of a pregnant woman, transmit a signal to an ultrasound device to request that imaging start, and acquire an image signal from the ultrasound device; based on that an amount of pixel change on an imaging screen corresponding to the image signal received from the ultrasound device is lower than a specified level, and that a corresponding interval is maintained for more than a specified time, determine that the imaging screen is in a paused state; take a snapshot on the imaging screen at a time when the imaging screen is determined to be in the paused state, and store the snapshot separately in the memory; based on the probe not moving until a first point in time after a specified time period from a point in time when the snapshot is taken, stop the imaging at the first point in time; based on the probe moving again, resume the imaging; based on a value of a grey scale on the imaging screen exceeding a specified level, determine that examination is over and transmit a signal to the ultrasound device to request that the imaging be terminated; and based on the imaging being terminated, store, in the memory, images captured from a moment of start of the imaging to a moment of termination of the imaging.

### Advantageous Effects

A system according to one embodiment may start recording from the moment the examiner's ultrasound probe contacts the subject to be examined after starting the examination, and detect the end of the examination and stop recording based on the grey scale and motion levels in the video, thereby reducing the size of the recorded video stored in memory.

By reducing the size of the recorded video stored in memory, the system according to one embodiment may reduce transmission time and costs, and may provide a user experience that makes it easier for the user to utilize or manage the video.

In one embodiment, the system may automatically edit and present meaningful segments of the fetal video to the user based on the examiner's motion and the grey scale and motion level of the captured images.

### DESCRIPTION OF DRAWINGS

FIG. 1A illustrates a situation in which an ultrasound imaging device according to one embodiment captures an ultrasound image and obtains user authentication information from a server.

FIG. 1B illustrates a probe of an ultrasound imaging device according to one embodiment.

FIG. 2 is a block diagram illustrating a configuration of a system for generating and editing ultrasound images according to one embodiment.

FIG. 3 is a flowchart illustrating a method of generating and editing ultrasound images by a system according to one embodiment.

FIG. 4 is a flow diagram of a method of generating and editing ultrasound images by a system according to one embodiment.

### BEST MODE

Hereinafter, embodiments are described in detail with reference to the accompanying drawings. However, various modifications may be made to the embodiments and the scope of the claims of the patent application is not limited or defined by these embodiments. It is to be understood that all modifications, equivalents, or substitutions to the embodiments are covered by the scope of the claims.

Any particular structural or functional description of the embodiments is disclosed for illustrative purposes only and may be modified and practiced in various forms. Accordingly, the embodiments are not limited to any particular disclosed form, and the scope of this disclosure includes any modifications, equivalents, or substitutions covered by the technical idea.

Terms such as first or second may be used to describe various components, but these terms should be interpreted only to distinguish one component from another. For example, a first component may be named as a second component, and similarly, a second component may be named as a first component.

When a component is referred to as being “connected” to another component, it is to be understood that it may be directly connected or coupled to the other component, or that there may be other components between the components.

The terms used in the embodiments are for illustrative purposes only and are not intended to be construed as limiting. A singular expression includes a plural expression unless the context clearly dictates otherwise. In the present disclosure, the term “include” or “have” is intended to indicate that characteristics, figures, steps, operations, constituents, and components disclosed in the specification or combinations thereof exist. The term “include” or “have” should be understood as not pre-excluding possibility of existence or addition of one or more other characteristics, figures, steps, operations, constituents, components, or combinations thereof.

Unless defined otherwise, all terms used herein, including technical or scientific terms, have the same meaning as commonly understood by one of ordinary skill in the art to which the embodiments pertain. Terms such as those defined in commonly used dictionaries should be interpreted as having a meaning that is consistent with their meaning in the context of the present disclosure and the relevant art and will not be interpreted in an idealized or overly formal sense unless expressly so defined herein.

In addition, in the description with reference to the accompanying drawings, the same components are assigned the same reference numerals regardless of the reference numerals, and the redundant description thereof will be omitted. In describing embodiments, a detailed description of a related known technology may be omitted to avoid obscuring the subject matter of the embodiments.

Embodiments can be implemented in various forms of products, including personal computers, laptop computers, tablet computers, smartphones, televisions, smart home appliances, intelligent vehicles, kiosks, and wearable devices.

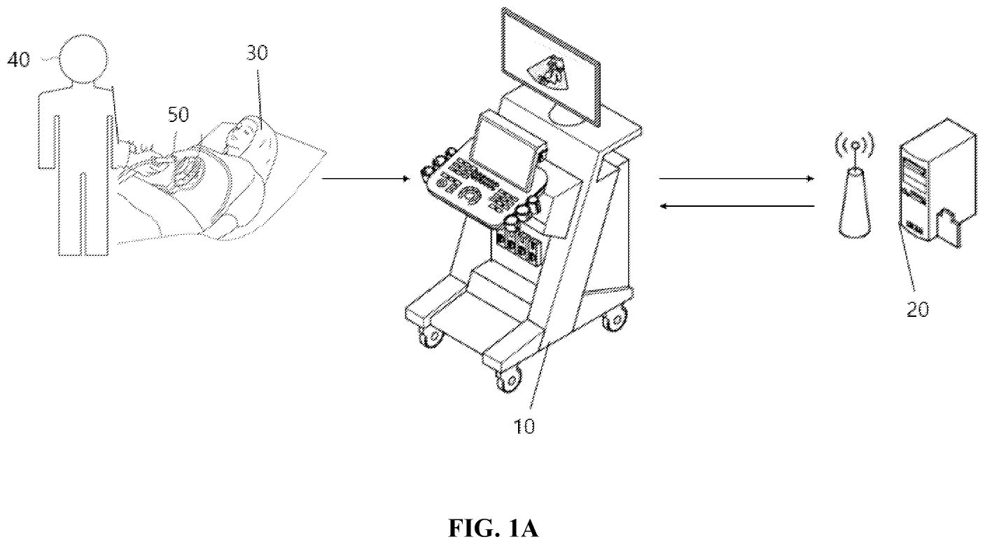

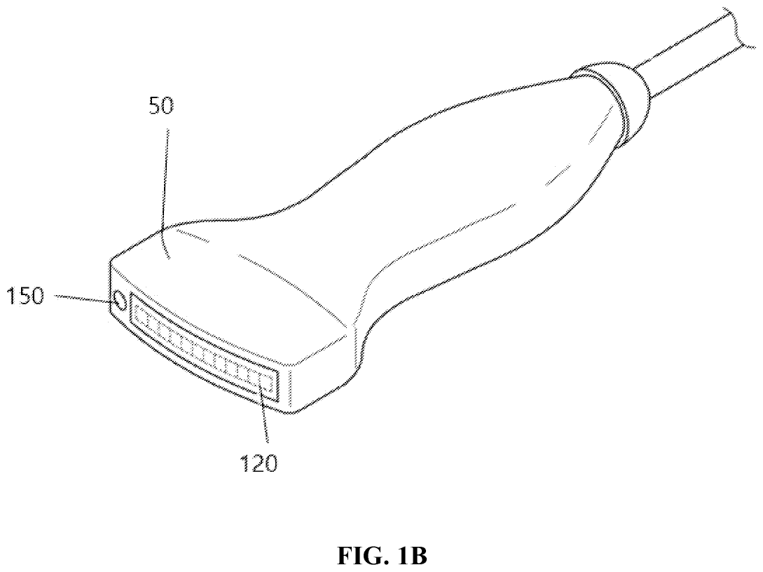

FIG. 1A illustrates a situation in which an ultrasound imaging device according to one embodiment captures an ultrasound image and obtains user authentication information from a server.

Referring to FIG. 1A, an electronic device (e.g., ultrasound imaging device) 10 of the system may receive from a user input of pregnancy-related information about a patient (e.g., a pregnant woman, a new mother). The pregnancy-related information may include at least one of a patient's last menstrual period (LMP) and a date of conception (DOC). The electronic device 10 may radiate an ultrasound signal to the abdomen of the patient 30 via the ultrasound probe 50, and may acquire an ultrasound image of the fetus upon receiving an ultrasound echo signal reflected from the patient 30. The electronic device 10 may measure the size of a body part of the fetus in the ultrasound image of the fetus. The electronic device 10 may receive membership information including an identifier from the server 20. The membership information may be determined differently for each patient 30 and may include at least one of a member name, a name of the fetus, the number of months of pregnancy, or a date of examination. The electronic device 10 may receive body measurement data about the fetus related to the pregnancy information from the server 20. The server 20 may collect the fetal body size information received from a plurality of different devices and store big data related to the fetal body measurements. In one embodiment, based on the big data related to the fetal body measurements, the server 20 may provide information about the patient 30 and the patient's fetus (e.g., the degree of growth compared to other fetuses, the fetus' biparietal diameter (BPD), abdominal circumference (AC), head circumstance (HC), occipitofrontal diameter (OFD), and femur length (FL)).

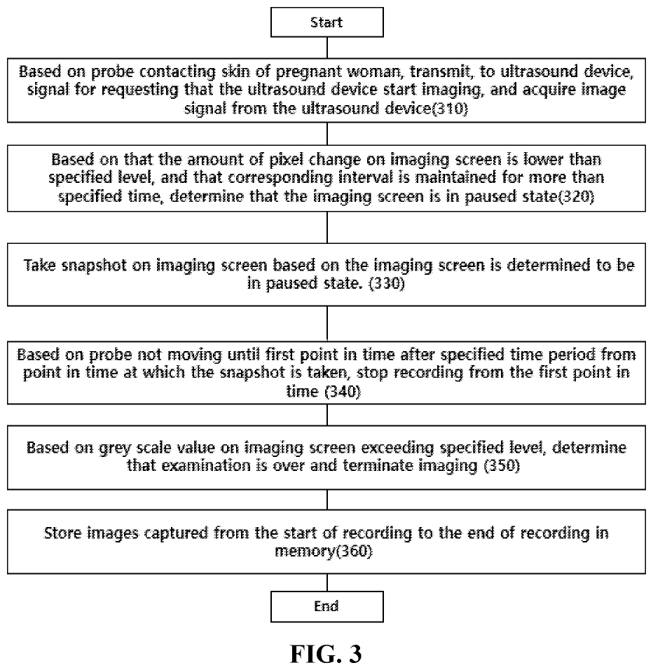

According to one embodiment, based on the examiner 40 bringing the probe 50 into contact with a body part of the patient 30, the electronic device 10 may start acquiring ultrasound images. The electronic device 10 may use the server 20 to acquire information about the patient 30 or information about the user, and may transmit the captured video and edited video or image to the user's terminal or the terminal of the patient 30. Capturing and editing of ultrasound images will be described with reference to FIG. 3.

FIG. 1B illustrates a probe of an ultrasound imaging device according to one embodiment.

According to FIG. 1B, an end of the probe 50 may be provided with a plurality of ultrasonic transducers 120, which generate ultrasound in response to an electrical signal, and a sample image acquisition portion 150. The ultrasonic transducers 120 may generate ultrasound in response to an applied alternating current power. The ultrasound transducers 120 may be supplied with alternating current power from an external power supply or an internal storage device, such as a battery. The piezoelectric pendulum or thin film or the like of the ultrasonic transducer 120 may vibrate in response to the supplied alternating current power to generate ultrasound. The ultrasonic transducer 120 may include, for example, any one of a magnetostrictive ultrasonic transducer utilizing the magnetostric tive effect of magnetic materials, a piezoelectric ultrasonic transducer utilizing the piezoelectric effect of piezoelectric materials, or a capacitive micromachined ultrasonic transducer transmitting and receiving ultrasonic waves using the vibration of hundreds or thousands of micromachined thin films. The ultrasonic transducers 120 may be arranged in a linear array or in a convex array.

The sample image acquirer 150 may acquire one or more sample images having different brightnesses by imaging the skin of the subject, i.e., the pregnant woman. Here, the sample images may refer to images containing information about the skin color or skin texture of the pregnant woman. The sample image acquirer 250 may be implemented as a camera module assembled with a lens, an image sensor, an IR filter (infrared cutoff filter), an actuator, and a flexible PCB (FPCB). In this case, sample images with different brightnesses can be acquired by controlling the exposure time of the camera module. Here, controlling the exposure time of the camera module may be performed during the ultrasound diagnosis. The sample image acquirer 150 may be implemented solely as an image sensor. The image sensor may include, for example, a complementary metal-oxide-semiconductor (CMOS) image sensor or a charge-coupled device (CCD) image sensor. The image sensor may include an external lens, a micro lens, a color filter array, a pixel array, an A/D converter that converts an analog signal received from the pixel array to a digital signal, and a digital signal processor that processes the digital signal output from the A/D converter.

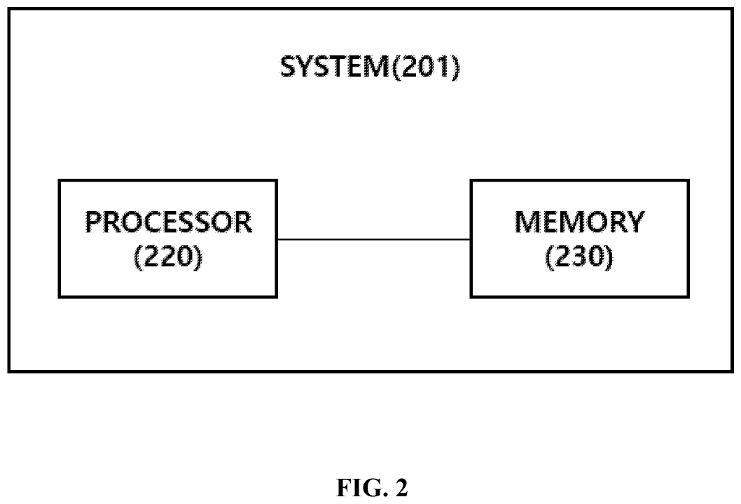

FIG. 2 is an exemplary diagram illustrating a configuration of a system for generating and editing ultrasound images according to one embodiment.

The system 201 according to an embodiment may include a processor 220 and a memory 230, and some of the illustrated components may be omitted or replaced. The system 201 according to one embodiment may be a server or a terminal. According to one embodiment, the processor 220 is a component capable of performing calculations or data processing related to control and/or communication of each component of the system 201, and may include one or more processors. The memory 230 may store information related to the above-described method or a program in which the above-described method is implemented. The memory 230 may be volatile memory or non-volatile memory. The memory 230 may store various file data, and the stored file data may be updated according to the operation of the processor 220.

According to one embodiment, the processor 220 may execute a program and control the system 201. Program codes executed by the processor 220 may be stored in the memory 230. Operations of the processor 220 may be performed by loading instructions stored in the memory 230. The system 201 may be connected to an external device (e.g., a personal computer or network) through an input/output device (not shown) and exchange data.

According to one embodiment, the system 201 may further include a microphone. The processor 220 may receive the strength of the audio output signal for the captured image using the microphone, and amplify the strength of the audio output signal for the captured image based on the strength of the audio output signal being lower than (or lower than or equal to) a specified level. Based on the intensity of the audio noise according to the external environment in the captured image exceeding a specified level, the strength of the audio output signal for the captured image may be further increased.

According to one embodiment, the processor 220 may amplify the strength of the audio output signal such that the strength of the audio output signal for the captured image has a value between −10 dB and 10 dB based on the strength of the audio output signal being lower than (or lower than or equal to) a specified level (e.g., −20 dB). When the maximum sound level in the ultrasound image is lower than (or lower than or equal to) a specified level (e.g., −20 dB), the processor 220 may amplify at least 10 dB to increase the volume to near 0 dB. The processor 220 may amplify the sound in the ultrasound image in a typical noisy environment (e.g., 0 to 40 dB) in which a user uses the terminal to help the user recognize the sound. The strength of the audio output signal and the strength of the amplified signal are exemplary, but not limiting.

According to one embodiment, based on the probe contacting the skin of the pregnant woman, the processor 220 transmits a signal to the ultrasound device to request that the ultrasound device start imaging, and acquires an image signal from the ultrasound device. Also, based on that the amount of pixel change on the imaging screen corresponding to the image signal received from the ultrasound device is lower than a specified level, and that the corresponding interval is maintained for more than a specified time, the processor 220 determines that the imaging screen is in a paused state, takes a snapshot on the imaging screen at the time when the imaging screen is determined to be in the paused state, and stores the snapshot separately in the memory 230. Then, based on the probe not moving until a first point in time after a specified time period from a point in time when the snapshot is taken, the processor stops the imaging at the first point in time. Based on the probe moving again, the processor resumes the imaging. Based on a grey scale value on the imaging screen exceeding a specified level, the processor determines that the examination is over and transmits a signal to the ultrasound device to request that the imaging be terminated. Based on the imaging being terminated, the processor stores the captured images from the moment of the start of the imaging to the moment of the end of the imaging in the memory 230.

According to one embodiment, based on recognizing an identifier marked on an external device, the processor 220 may determine that a user of the external device has entered the examination room, and establish a communication connection with the external device. Based on that the imaging is terminated, the processor 220 may transmit, to the external device recognized by the identifier, the snapshot stored in the memory 230 and the image taken from the moment of the start of the imaging to the moment of the end of the imaging. The identifier may include at least one of a QR code, radio frequency identification (RFID), near field communication (NFC), or barcode.

Upon completion of the imaging, the processor 220 may automatically send the snapshot and captured images to the user (e.g., the pregnant woman) to provide convenience to the user.

According to one embodiment, the computational and data processing functions that can be implemented by the processor 220 on the system 201 is not limited. Hereinafter, a detailed description will be given of the function of starting imaging from the moment the examiner's ultrasound probe contacts a subject after start of the examination, detecting that the examination is over based on the grey scale and motion level of the images, and stop the imaging to reduce the size of the captured images to be stored in the memory.

FIG. 3 is a flowchart illustrating a method of generating and editing ultrasound images by a system according to one embodiment.

The operations described with reference to FIG. 3 may be implemented based on instructions that may be stored on a computer recording medium or memory (e.g., the memory 230 of FIG. 2). The illustrated method may be implemented by the system previously described with reference to FIGS. 1A to 2 (e.g., the system 201 of FIG. 2). Technical features previously described will be omitted herein. The order of the respective operations in FIG. 3 may be changed. Some of the operations may be omitted, and some of the operations may be performed simultaneously.

In operation 310, based on a probe (e.g., the probe 50 of FIG. 1A) contacting the skin of a pregnant woman, the processor (e.g., the processor 220 of FIG. 2) may transmit, to the ultrasound device, a signal for requesting that the ultrasound device start imaging, and acquire an image signal from the ultrasound device.

In operation 320, based on that the amount of pixel change on the imaging screen is lower than a specified level, and that the corresponding interval is maintained for more than a specified time, the processor 220 may determine that the imaging screen is in a paused state.

In one embodiment, the processor 220 may determine that the imaging screen is in the paused state based on that the amount of pixel change on the imaging screen is lower than about 5%, and that the corresponding interval during which the amount of pixel change on the imaging screen is lower than the specified level (e.g., 5%) exceeds a specified time (e.g., 3 seconds).

In one embodiment, the processor 220 may classify the values of the pixels of the captured images into lightness, chroma, and hue, compute a shade value and a depth value based on the lightness, chroma, and hue of the pixels of the captured images, determine the gray scale of the captured images based on the shade value, and determine an amount of change of the pixels of the captured images based on the shade value and the depth value.

In operation 330, the processor 220 may take a snapshot on the imaging screen at a time when the imaging screen is determined to be in the paused state.

In operation 340, based on the probe not moving until a first point in time after a specified time period from the point in time at which the snapshot is taken, the processor 220 may stop imaging from the first point in time.

In one embodiment, the first point in time may be when the examiner has stopped moving the probe (e.g., the probe 50 in FIG. 1B) to explain a particular situation to the pregnant woman, but not when the imaging has ended. Since the first point in time is intended for the examiner to describe a particular situation to the pregnant woman, it may be a situation in which the fetus is moving or in which there is a significant change in the fetus. The processor 220 may capture a snapshot of the imaging screen at the first point in time, store the snapshot separately in the memory 230, and deliver the snapshot to the user (e.g., the pregnant woman). The processor 220 may provide the user with a compilation of only relatively significant moments for the fetus to view, thereby saving time, reducing communication costs, and providing ease of use in editing the images, compared to the case where the user needs to check all of the ultrasound images.

In one embodiment, based on recognizing an identifier marked on an external device, the processor 220 may determine that a user of the external device has entered the examination room, and establish a communication connection with the external device. Based on the imaging being terminated, the processor 220 may transmit, to the external device recognized by the identifier, the snapshot stored in the memory 230 and the images taken from the moment of the start of the imaging to the moment of the end of the imaging. The identifier may include at least one of a QR code, radio frequency identification (RFID), near field communication (NFC), or barcode.

In one embodiment, based on recognizing the identifier marked on the external device, the processor 220 may display a first parenting message corresponding to the user's entry into the examination room. Based on the probe 50 contacting the skin of the pregnant woman, the processor 220 may display a second parenting message at a moment when imaging starts, and display a third parenting message at a time when the imaging screen is determined to be paused. Based on a grey scale value on the imaging screen exceeding a specified level, the processor 220 may display a fourth parenting message at a time when the imaging is terminated. For example, the first parenting message may include a time and date when the pregnant woman enters the examination room. The second parenting message may include a message indicating that imaging is to start. The third parenting message may include a message indicating a significant moment. The fourth parenting message may include a message indicating that the imaging has ended.

In one embodiment, the processor 220 may identify, by the date of capture, at least one snapshot taken at the moment when it is determined that the imaging is paused, and may indicate, on a timestamp, a point in time including the at least one snapshot within the images captured from the moment when the imaging starts to the moment when the imaging ends. The processor 220 may assemble the at least one snapshot and create a separate footage, add a preset message or special effects, and send the footage to an external device (e.g., a user terminal).

In operation 350, the processor 220 may determine whether the examination has been completed based on a grey scale value on the imaging screen.

In one embodiment, the processor 220 may determine that the examination is over based on a grey scale value on the imaging screen exceeding 70%. Based on the determination that the examination is over, the processor 220 may display, on the display (not shown), information indicating that the examination is over and that the recording of the imaging footage is complete. Based on the determination that the examination is over, the processor 220 may transmit to the external device (e.g., the user terminal) information indicating that the examination is over and the recording of the imaging footage is complete.

In operation 360, based on determining that the imaging has ended, the processor 220 may store the images captured from the moment of the start of the imaging to the moment of the end of the imaging in the memory.

Based on determining that the imaging has ended, the processor 220 may transmit, to the external device, the snapshot stored in the memory 130 and the images captured from the moment of the start of the imaging to the moment of the end of the imaging. When the imaging ends, the processor 220 may automatically send the snapshot and captured images to the user (e.g., the pregnant woman) to provide convenience to the user.

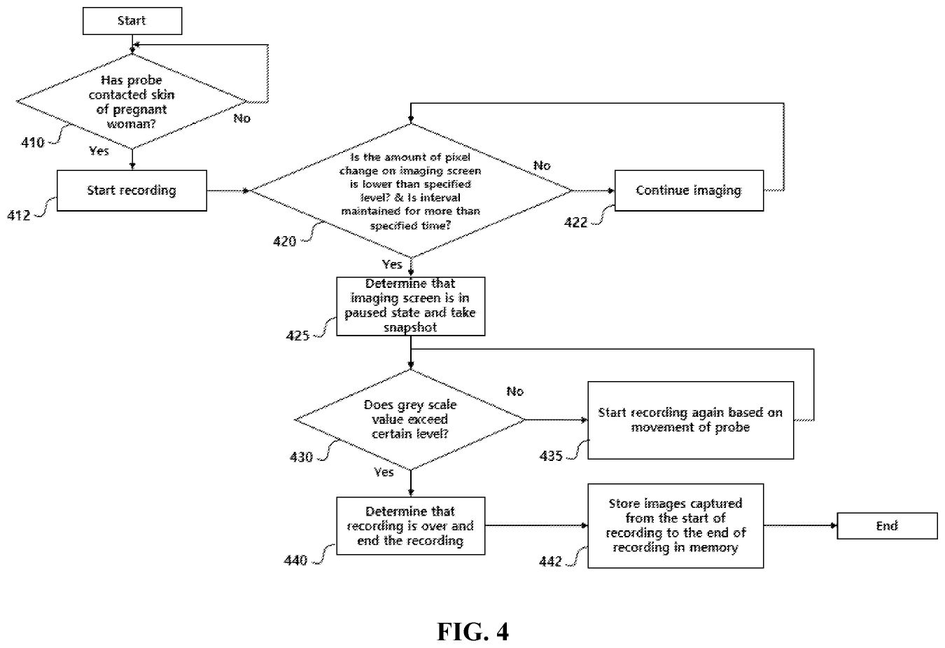

FIG. 4 is a flow diagram of a method of generating and editing ultrasound images by a system according to one embodiment.

The operations described with reference to FIG. 4 may be implemented based on instructions that may be stored on a computer recording medium or memory (e.g., the memory 230 of FIG. 2). The illustrated method may be implemented by the system previously described with reference to FIGS. 1A to 2 (e.g., the system 201 of FIG. 2). Technical features previously described will be omitted herein. The order of the respective operations in FIG. 4 may be changed. Some of the operations may be omitted, and some of the operations may be performed simultaneously.

In operation 410, the processor (e.g., the processor 220 of FIG. 2) may determine whether the probe (e.g., the probe 50 of FIG. 1A) has contacted the skin of the pregnant woman (or patient). When the probe 50 has not contacted the skin of the pregnant woman, the processor 220 may continue to detect whether the probe 50 has contacted the skin of the pregnant woman without starting imaging.

In operation 412, based on the probe 50 contacting the skin of the pregnant woman, the processor 220 may start imaging. Based on the movement of the probe 50, the processor 220 may determine a moment at which imaging starts, and may start imaging to reduce the length of the footage.

In operation 420, the processor 220 may determine whether the amount of pixel change on the imaging screen is lower than a specified level, and whether the corresponding interval in which the amount of pixel change is lower than the specified level is maintained for more than a specified time.

In one embodiment, the processor 220 may determine that the imaging screen is in a paused state based on that the amount of pixel change on the imaging screen is lower than (or lower than or equal to) a specified level (e.g., about 5%), and that the corresponding interval during which the amount of pixel change on the imaging screen is lower than the specified level (e.g., about 5%) exceeds a specified time (e.g., about 3 seconds). When the imaging continues to be recorded while the imaging screen is paused, the length of the recorded ultrasound images may increase and take up a relatively large amount of space in the memory 230. In addition, the user may find it difficult to identify significant moments (e.g., a moment when the fetus moves, a moment when an appearance that is different from the previous one is observed) due to the excessive length of the footage. A system for generating and editing ultrasound images according to the present disclosure may stop recording upon detecting a pause in the imaging screen, thereby reducing the overall length of the footage, while still capturing and separately presenting to the user an image of the fetus at a significant moment when the examiner stops the probe and explains something to the user (e.g., the pregnant woman).

In one embodiment, the processor 220 may classify the values of the pixels of the captured image into lightness, chroma, and hue, compute a shade value and a depth value based on the lightness, chroma, and hue of the pixels of the captured image, determine a grey scale of the captured image based on the shade value, and determine an amount of change of the pixels of the captured image based on the shade value and the depth value.

In operation 422, based on that the interval in which the amount of pixel change on the imaging screen exceeds a certain level or the interval in which the amount of pixel change on the imaging screen is lower than the certain level fails to exceed a specified time, the processor 220 may continue imaging. When the amount of pixel change on the imaging screen exceeds the certain level, the processor 220 may determine to continue imaging with the probe 50 moving again, and may resume imaging rather than stopping imaging. When the interval in which the amount of pixel change on the imaging screen is lower than the certain level does not exceed the specified time (e.g., 3 seconds), the processor 220 may determine that the examiner has not stopped moving the probe 50 and may continue imaging.

In operation 425, based on that the amount of pixel change on the imaging screen is lower than a specified level, and that the corresponding interval is maintained for more than the specified time, the processor 220 may determine that the imaging screen is in a paused state. Upon determining that the imaging screen is in the paused state, the processor 220 may stop recording, thereby reducing the length of the recorded ultrasound images (or images of the fetus). Additionally, the processor 220 may take a snapshot of the imaging screen at the time it determines that the imaging screen is in the paused state and store the snapshot in the memory 230. The transition from imaging in progress to the paused state is intended to allow the examiner to explain the imaging screen to the pregnant woman, and the imaging screen may correspond to a significant moment that requires explanation to the pregnant woman. The processor 220 may take a snapshot of the imaging screen at the time it determines that the imaging screen is in the paused state, and store the snapshot separately in the memory 230 to provide the snapshot to the pregnant woman later.

In operation 430, the processor 220 may classify the values of the pixels of the captured image into lightness, chroma, and hue, compute a shade value and a depth value based on the lightness, chroma, and hue of the pixels of the captured image, and determine a grey scale of the imaging screen based on the shade value.

In operation 435, based on the grey scale value being lower than the certain level, the processor 220 may determine that the imaging is not over. The processor 220 may then start imaging again based on the movement of the probe (e.g., the probe 50 of FIG. 1A).

In operation 440, based on the grey scale value exceeding the certain level, the processor 220 may determine that the imaging is over and terminate the imaging (or recording). By terminating the imaging, the processor 220 may prevent unnecessary portions from being recorded after the end of the examination, thereby preventing the length of the footage from increasing.

In operation 450, the processor 220 may store, in the memory 130, the images captured from the moment of the start of the imaging to the moment of the end of the imaging. The processor 220 may automatically send the captured images to the registered user.

In one embodiment, the processor 220 may identify, by the date of capture, at least one snapshot taken at the moment when it is determined that the imaging is paused, and may indicate, by a timestamp, the point in time that includes the at least one snapshot within the images captured from the moment when the imaging starts to the moment when the imaging ends. The processor 220 may assemble the at least one snapshot and create a separate footage, add a preset message or special effects, and send the footage to an external device (e.g., a user terminal).

According to one embodiment, based on that the amount of pixel change on the imaging screen is lower than 5%, and that the interval in which the amount of pixel change on the imaging screen is lower than 5% exceeds 3 seconds, the processor 220 may determine that the imaging screen is in the paused state. Also, based on the grey scale value on the imaging screen exceeding 70%, the processor 220 may determine that the examination is over.

According to one embodiment, the processor 220 may receive the strength of the audio output signal for the captured image using the microphone, and amplify the strength of the audio output signal for the captured image based on the strength of the audio output signal being lower than (or lower than or equal to) a specified level. Based on the intensity of the audio noise according to the external environment in the captured image exceeding a specified level, the strength of the audio output signal for the captured image may be further increased.

According to one embodiment, based on the strength of the audio output signal being lower than (or lower than or equal to) −10 dB, the processor 220 may amplify the strength of the audio output signal such that the strength of the audio output signal for the captured images has a value between −10 dB and 10 dB.

According to one embodiment, based on determining that the imaging is over, the processor 220 may display, on the display, information indicating that the examination is over and that the recording of the captured images is complete.

According to one embodiment, based on the determination that the imaging is over, the processor 220 may transmit, to an external device, the information indicating that the examination is over and that the recording of the captured images is complete.

The embodiments described above may be implemented by hardware components, software components, and/or a combination of hardware components and software components. For example, the apparatus, method, and components described in the embodiments may be implemented using one or more general purpose or special purpose computers such as, for example, a processor, a controller, an arithmetic logic unit (ALU), a digital signal processor, a microcomputer, a field programmable gate array (FPGA), a programmable logic unit (PLU), microprocessors, or any other device capable of executing and responding to instructions. A processing unit may run an operating system (OS) and one or more software applications executed on the OS. The processing unit may also access, store, manipulate, process, and generate data in response to execution of software. While it is described for convenience of understanding that one processing unit is used, those skilled in the art will understand that the processing unit may include a plurality of processing elements and/or a plurality of types of processing elements. For example, the processing unit may include a plurality of processors or one processor and a controller. Other processing configurations such as parallel processors are also possible.

The method according to the embodiment may be implemented in the form of program instructions that may be executed through various computer means and recorded on a computer-readable medium. The computer-readable medium may include program instructions, data files, and data structures alone or in combination. The program instructions recorded on the medium may be specially designed and configured for the embodiments or may be known and available to those skilled in computer software. Examples of the computer-readable recording media include magnetic media such as hard disks, floppy disks and magnetic tapes, optical media such as CD-ROMs and DVDs, magneto-optical media such as floptical disks, and hardware devices specially configured to store and execute program instructions, such as ROM, RAM, and flash memory. Examples of program instructions include high-level language codes that may be executed by a computer using an interpreter, as well as machine language codes such as those produced by a compiler. The hardware devices described above may be configured to operate as one or more software modules to perform the operations of the embodiments, and vice versa.

Software may include a computer program, code, instructions, or a combination of one or more of the foregoing, and may configure a processing unit to operate as desired or independently or collectively instruct the processing unit. Software and/or data may be permanently or temporarily embodied in any type of machine, component, physical device, virtual equipment, computer storage medium or device, or a transmitted signal wave in order to be interpreted by or provide instructions or data to a processing unit. Software may be distributed on networked computer systems and may be stored or executed in a distributed manner. The software and data may be stored on one or more computer-readable media. As disclosed above, the embodiments have been described by limited drawings.

However, those skilled in the art may apply various technical modifications and variations based on the above. For example, the described techniques may be carried out in an order different from the method described, and/or components of the described system, structure, apparatus, circuit, and the like may be coupled or combined in a different form than the method described, or replaced or substituted by other components or equivalents that may achieve appropriate results.

Therefore, other implementations, other embodiments, and equivalents of the claims are also within the scope of the accompanying claims.

1. A system for generating and editing ultrasound images, the system comprising:
a memory; and
a processor,
wherein the processor is configured to:
based on a probe contacting a skin of a pregnant woman, transmit a signal to an ultrasound device to request that imaging start, and acquire an image signal from the ultrasound device;
based on that an amount of pixel change on an imaging screen corresponding to the image signal received from the ultrasound device is lower than a specified level, and that a corresponding interval is maintained for more than a specified time, determine that the imaging screen is in a paused state;
take a snapshot on the imaging screen at a time when the imaging screen is determined to be in the paused state, and store the snapshot separately in the memory;
based on the probe not moving until a first point in time after a specified time period from a point in time when the snapshot is taken, stop the imaging at the first point in time;
based on the probe moving again, resume the imaging;
based on a value of a grey scale on the imaging screen exceeding a specified level, determine that examination is over and transmit a signal to the ultrasound device to request that the imaging be terminated; and
based on the imaging being terminated, store, in the memory, images captured from a moment of start of the imaging to a moment of termination of the imaging.

2. The system of claim 1, wherein the processor is configured to:
based on recognizing an identifier marked on an external device, determine that a user of the external device has entered an examination room, and establish a communication connection with the external device; and
based on the imaging being terminated, transmit, to the external device recognized by the identifier, the snapshot stored in the memory and the images captured from the moment of start of the imaging to the moment of termination of the imaging,
wherein the identifier comprises at least one of a QR code, radio frequency identification (RFID), near field communication (NFC), or barcode.

3. The system of claim 2, wherein the processor is configured to:
based on recognizing the identifier marked on the external device, display a first parenting message corresponding to the user's entry into the examination room; and
based on the probe contacting the skin of the pregnant woman, display a second parenting message at the moment of the start of the imaging;
display a third parenting message at a moment when the imaging screen is determined to be in the paused state; and
based on the value of the grey scale on the imaging screen exceeding the specified level, display a fourth parenting message at a time when the imaging is terminated.

4. The system of claim 1, wherein the processor is configured to:
based on that the amount of pixel change on the imaging screen is lower than 5%, and that the interval in which the amount of pixel change on the imaging screen is lower than 5% exceeds 3 seconds, determine that the imaging screen is in the paused state; and
based on the value of the grey scale on the imaging screen exceeding 70%, determine that the examination is over.

5. The system of claim 1, wherein the processor is configured to:
classify values of pixels of the captured images into lightness, chroma, and hue;
compute a shade value and a depth value based on the lightness, chroma, and hue of the pixels of the captured images;
determine the gray scale of the captured images based on the shade value; and
determine an amount of change of the pixels of the captured images based on the shade value and the depth value.

6. The system of claim 1, further comprising:
a microphone,
wherein the processor is configured to:
receive a strength of an audio output signal for the captured images using the microphone;
based on the strength of the audio output signal being lower than (or lower than or equal to) a specified level, amplify the strength of the audio output signal for the captured images; and
based on an intensity of audio noise according to an external environment in the captured images exceeding a specified level, increase the strength of the audio output signal for the captured images.

7. The system of claim 6, wherein, based on the strength of the audio output signal being lower than (or lower than or equal to) −10 dB, the processor amplifies the strength of the audio output signal such that the strength of the audio output signal for the captured images has a value between −10 dB and 10 dB.

8. The system of claim 1 wherein, based on determining that the imaging is over, the processor displays, on a display, information indicating that the examination is over and that recording of the captured images is complete.

9. The system of claim 1, wherein, based on determining that the imaging is over, the processor transmits, to an external device, information indicating that the examination is over and that recording of the captured images is complete.

10. The system of claim 1, wherein the processor is configured to:
identify, by a date of capture, at least one snapshot taken at a moment when it is determined that the imaging is in the paused state;
indicate, on a timestamp, a point in time including the snapshot within the images captured the moment of start of the imaging to the moment of termination of the imaging;
assemble the at least one snapshot and create a separate footage; and
add a preset message or special effects to the footage and transmit the footage to an external device.
